(19) United States
(12) Patent Application Publication
(10) Pub. No.: US 2026/0272347 A1
(43) Pub. Date: Sep. 17, 2026

(54) INTRAVASCULAR BLOOD EXTRACTION SYSTEMS, DEVICES, AND METHODS

(71) Applicant: VenoCare, Inc., Doral, FL (US)

(72) Inventors: Raul LEYTE-VIDAL, Doral, FL (US); Amir BELSON, Savyon (IL); Daniel PICO, Doral, FL (US); Paul FENTON, Doral, FL (US); Ryan PATTERSON, Kaysville, UT (US)

(21) Appl. No.: 19/165,093

(22) PCT Filed: Mar. 14, 2024
(86) PCT No.: PCT/US2024/020028

§ 371 Date: Sep. 12, 2025

Related U.S. Application Data
(60) Provisional application No. 63/490,242, filed on Mar. 14, 2023.

(51) Int. Cl. A61B 5/15

(52) U.S. Cl. CPC A61B 5/15003; A61B 5/150221; A61B 5/150992

(57) ABSTRACT

An intravascular blood extraction system for phlebotomy through a previously placed peripheral intravascular catheter having a cannula within a protective housing and/or structures and/or structure, a cannula forming a laminar fluid path extending from the distal fluid entry to a lateral or proximal fluid exit orifices directly coupled to the protective housing and/or structures and/or structure, a slider o plunger actuator coupled to the fluid exit orifice of the cannula so that distal advancement of the slider advances the cannula inside a fluid vessel.

### CLAIM OF PRIORITY

This application claims the benefit of U.S. Provisional Patent Application No. 63/490,242, filed Mar. 14, 2023, and titled “INTRAVASCULAR BLOOD EXTRACTION SYSTEMS, DEVICES, AND METHODS,” which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Blood extraction is a necessary element for several medical procedures and/or therapies. The fluid sampling is usually performed by venipuncture using a sharp needle to obtain intravenous access.

While outpatient venipuncture and other forms of vascular access in relatively healthy patients can be a simple matter, such access is often needed in patients who are admitted into a healthcare institution for multiple days thus experiencing multiple venipunctures. In general, Blood extraction via venipuncture is the most invasive procedure for inpatients, who experience an average two or more fluid collection episodes a day. Although venipuncture is common and provides high-quality fluid samples, is associated with high patient dissatisfaction due to needlestick pain and other complications such as lack of sleep, infection, nerve damage, inadvertent arterial puncture, thrombus, vessel depletion, and hematomas. In addition, needle handling can increase the risk of accidental needlestick injury for providers. Lastly difficult IV access patients register multiple failed attempts which lead to significant clinical delays, lost productivity, and dissatisfaction for patients and providers.

A possible alternative to venipunctures is the use of peripheral intravenous catheters (PIVCs). PIVCs are placed in the majority of admitted patients but there are a number of factors reducing the likelihood of success for this procedure. Fluid collected via a PIVC must meet a number of requirements for accurate laboratory analysis, such as an acceptable fluid volume and quality, samples cannot be hemolyzed, clotted, contaminated, and/or diluted. Frequently, initial fluid draws must be discarded to remove potential contaminants from the fluid sample. If the quality of fluid is not appropriate, analytical tests cannot be performed and a re-draw is required. Re-draws also lead to significant clinical delays, loss of productivity, and dissatisfaction for patients and providers. Additionally, fluid sampling through a PIVC increases the risk of hemolysis (destruction of red fluid cells), contamination of the sample from infusing solutions and medications within the previously placed peripheral catheter, localized catheter kinks from excessive catheter movement, and dislodgment of the catheter out of the vasculature. Furthermore, fluid draw success through a PIVC is reduced as the likelihood of occlusion and/or catheter damage increases with extended catheter dwell times. Lastly, there's a risk of fluid stream infection if aseptic techniques are not utilized when accessing a previously placed peripheral catheter.

For these reasons, it would be desirable to provide improved methods and systems for needleless Blood extraction using previously placed peripheral catheters and structures. It would be particularly desirable to provide simplified systems and assemblies to remedy, reduce, or avoid one or more of the herein mentioned drawbacks. Even more desirably, to provide components which are clearly visible to the user and configured to be utilized and manipulated in a straightforward, intuitive manner. At least some of these objectives will be met by the various embodiments that follow.

### SUMMARY OF THE DISCLOSURE

Described herein are systems, devices, and methods for phlebotomy through a previously placed peripheral intravascular catheter.

The present disclosure relates generally to vascular blood extraction systems, devices, and methods. Particularly, in some embodiments, the present disclosure relates to systems, devices, and methods for placing a cannula within a previously placed peripheral intravascular catheter. The cannula is used to transfer a small volume of fluid from the patient to a fluid collection container coupled to the intravascular blood extraction system.

The blood extraction system can include systems or devices couplable with a previously placed peripheral intravascular catheter positioned in a blood vessel of a patient. The systems may include cannulas within flexible, and/or collapsible protective sheaths and/or structures to maintain sterility of the cannula during the blood extraction procedure, housing and/or structures, sliders, and mechanisms of coupling to a previously placed peripheral catheter. The cannulas may comprise of a single or a plurality of materials to increase its column strength. The sliders move the cannulas from a first position within the protective housing and/or structures to a distal position outside the protective housing and/or structures. A distal end portion of the housing and/or structures may be configured with mechanisms of coupling the blood extraction system to a previously placed peripheral intravascular catheter or similar connector and/or connection. The slider may be coupled to the housing and/or structures and/or coupled to the cannula and disposed outside and inside of the housing and/or structures of the device.

In some embodiments, a blood extraction system may allow a user to draw a fluid sample through a previously placed peripheral intravascular catheter. The cannula of the blood extraction system is advanced distally within the inner diameter of the catheter thus creating a laminar fluid path within the previously placed catheter and the blood extraction device. This laminar fluid path allows for a successful fluid draw from patients' vasculature into a fluid collection device coupled to the blood extraction system.

In some examples, the laminar fluid path of the blood extraction system may comprise of a distal fluid orifice located at the distal end of the cannula and a lateral fluid orifice located on a lateral surface of the intravascular blood extraction housing and/or structures. The lateral orifice may be comprised of a connector capable to facilitate the coupling of fluid collection devices (e.g., Vacutainer). Whereas the location of the lateral orifice of the laminar fluid path and connectors represent a more ergonomic way to attach fluid collection devices.

In some examples, the laminar fluid path of the blood extraction system may comprise of a distal entry located at the distal end of the cannula and a proximal exit located near the proximal end of the cannula. Whereas the proximal exit contains an orifice coupled, engaged, or located within a fluid transfer chamber directly coupled to the distal lateral orifice of the laminar fluid path. Whereas the distal entry and proximal exit orifices comprise the laminar fluid path within the cannula. A proximal exit orifice, as described herein, located distal to the slider distal end. A cannula, as described herein, having a proximal exit orifice to allow fluid to follow the laminar fluid path and exit into a fluid collection chamber. The fluid collection chamber, as described herein, is sealed around the cannula using polymeric seals, valves, or gaskets. Whereas the fluid collection chamber is directly coupled to the housing. Whereas the seals of the fluid collection chamber prevent leakage of fluid or vacuum pressure decay during use. The seals, as described herein, may be positioned within the fluid transfer chamber to form a seal around the cannula. Whereas the position or separation of these seals, valves, or gaskets may allow for the cannula proximal orifice to move into position within the fluid transfer chamber.

The cannula may be directly coupled to the slider and within a flexible and/or collapsible protective sheath. The flexible and/or collapsible protective sheath, as described herein, may be connected to the distal end of the housing and/or structures, and directly coupled to the distal end of the slider. The slider moves the cannula from a first position within the protective sheath and housing to a distal position outside the protective housing and/or structures. The flexible and/or collapsible protective sheath in the first position, may appear expanded, and contracted in the second position. The flexible and/or collapsible protective sheath maintains the sterile barrier around the cannula at all times.

In some embodiments, the laminar fluid path of the blood extraction system may comprise of a distal entry located at the distal end of the cannula and a lateral exit located distal to the slider. Whereas the slider lateral exit may be coupled to the distal lateral orifice of the protective housing and/or structures thus forming a laminar fluid path between the distal entry point on the cannula. A cannula, as described herein, having a proximal exit orifice to allow fluid to follow the laminar fluid path and exit into a fluid collection chamber. The exit orifice of the slider, as described herein, having seals, valves, or gasket connections may open when the slider is coupled to the structures comprising the lateral orifice. The seals coupled to the slider prevent leakage of fluid or vacuum pressure decay during use. The fluid path may comprise of a plurality of inner diameters through the fluid path. The structures of the lateral orifice may allow for the slider exit orifice to move into position and couple to the lateral orifice of the laminar fluid path located on the protective housing and/or structures.

The cannula may be directly coupled to the slider and within a flexible and/or collapsible protective sheath. The flexible and/or collapsible protective sheath, as described herein, may be connected to the distal end of the housing and/or structures, and directly coupled to the distal end of the slider. The slider moves the cannula from a first position within the protective housing and/or structures to a distal position outside the protective housing and/or structures. The flexible and/or collapsible protective sheath in the first position, as described herein, may appear expanded in the first position and contracted in the second position. The flexible and/or collapsible protective sheath maintains the sterile barrier around the cannula at all times.

In some embodiments, the cannula is protected by a collapsible and flexible sheath disposed within the housing and/or structures of the intravascular blood extraction device. The collapsible and flexible sheath may have a distal coupled to the protective housing and/or structures and proximal end coupled to the distal end of the slider. The slider moves the cannula from a first position within the protective housing and/or structures to a distal position outside the protective housing and/or structures. The flexible and/or collapsible protective sheath in the first position, as described herein, may appear expanded in the first position and contracted in the second position. The flexible and/or collapsible protective sheath maintains the sterile barrier around the cannula at all times.

In some examples, a fluid transfer tube is directly connected to the proximal end of the slider and coupled to the lateral orifice of the blood extraction device thus forming a laminar fluid path between the distal end and the lateral orifice of the blood extraction system. The fluid transfer tube, as described herein, may comprise of a flexible tube, and be coiled within the flexible and/or collapsible protective sheath.

In some example, the fluid transfer tube may be comprised of a single, non-coiled flexible tube disposed within the housing and/or structures of the blood extraction device. The flexible tube is coupled to the proximal end of the slider and allowed to move with the slider from a first position to a second position.

In some embodiments, the cannula is directly coupled to a plunger actuator. A plunger actuator may comprise of an exit port connected through a flexible transfer tube to the lateral orifice of the blood extraction system. In this example a fluid transfer tube is directly connected to the plunger actuator exit port and coupled to the lateral orifice of the blood extraction device thus forming a laminar fluid path between the distal end and the lateral orifice of the blood extraction system. The fluid transfer tube, as described herein, may comprise of a flexible tube, and be coiled within the flexible and/or collapsible protective sheath. The flexible and/or collapsible protective sheath maintains the sterile barrier around the cannula at all times.

In some embodiments, the cannula comprises of a polymeric and/or metallic stiffening structure along the length of the cannula. The stiffening structure can be oriented co-axially and/or circumferentially to the cannula and extend the entire length of the cannula or located in a segment or segments therein. The stiffening structure may include a multilayer tube and methods of making the same, whereas the multilayer tube comprises a first inner polymer layer and a second outer polymer layer, wherein the first and second polymer layer the stiffening structure is found.

In some examples, the inner layer of the cannula may be comprised of a hydrophobic material or materials. The single or a plurality of materials may be PTFE or similar to facilitate the transfer of fluids from the vasculature to the fluid collection device coupled to the extraction device.

In one aspect, there is provided an intravascular blood extraction system having a cannula with a laminar fluid path, the cannula extending from the distal fluid entry orifice to the lateral exit orifice disposed within the protective handle, housing and/or structures. The laminar fluid path has a distal fluid entry orifice and lateral fluid exit orifice, the laminar fluid path extending proximally through one or a plurality of cavities within the protective housing and/or structures and/or structures to the lateral fluid exit orifice of the laminar fluid path. The fluid exit orifice comprising of a means to connect a fluid collection device. There is also a slider or plunger actuator coupled to the fluid exit orifice of the cannula so that distal advancement of the slide advances the cannula from the distal end of the handle, protective housing and/or structures. The handle, protective housing and/or structures having a distal end and proximal end with a distal mechanism to couple the device with a previously placed peripheral catheter. There is also in some embodiments a flexible and/or collapsible sheath comprising of a means to isolate the cannula from external contamination. In other aspects, the intravascular blood extraction device has the cannula coupled to the distal end of the slider or the distal end of a plunger actuator, or the cannula is reinforced with a single or a plurality of materials. In another aspects, the cannula has a laminar fluid path with a fluid path sealer coupled to the proximal end of the housing to prevent backflow through the proximal orifice.

In this and other embodiments, the cannula is sealed and protected from contamination with the use of a flexible and/or collapsible sheath, optionally, the laminar fluid path has a plurality of orifices and cavities within the protective housing and/or structures, optionally, the laminar fluid path comprises of hydrophobic materials, optionally, the laminar fluid path comprises of flexible and/or collapsible transfer tubes disposed within the protective housing and/or structures. In still other embodiments, the laminar fluid path extends from the distal entry orifice of the cannula to the lateral exit orifice of the protective housing and/or structures.

Additionally or optionally, the laminar fluid path extends from a distal entry orifice of the cannula to the proximal exit orifice of the protective housing and/or structures. In additional aspects, the slider is positioned partially within the protective housing and/or structures and/or is movable within the boundaries of the protective housing and/or structures to move the cannula from a first position inside the protective housing and/or structures to a second position outside of the handle, the protective housing and/or structures. In additional aspects, the slider has a fluid path that directly couples to the proximal fluid exit orifice of the cannula and extends laterally to the lateral fluid exit orifice of the protective housing and/or structures, or the slider has a fluid path that directly couples to the proximal fluid exit orifice of the cannula and extends to the fluid path exit orifice of the slider when uncoupled to the lateral exit orifice of the protective housing and/or structures, or the fluid path exit orifice of the slider is sealed when uncoupled to prevent fluid leakage and/or pressure decay loss. In yet other aspects, the intravascular blood extraction device has a fluid path exit orifice of the slider is opened when coupled to the lateral exit orifice of the protective housing and/or structure with the use of seals, valves, gaskets, or other mechanisms. In still other alternatives, the protective housing and/or structures is comprised of seals, valves, gaskets, or other mechanisms to prevent fluid leakage and/or pressure decay around the cannula, chambers, and/or other components within the protective housing and/or structures.

In other aspects there is provided a method for collecting blood from an existing in-dwelling catheter or other placed vascular access component by coupling a blood extraction device to a previously placed peripheral catheter at least partially disposed within a fluid path of a patient. The blood extraction device includes at least a cannula with a distal entry and proximal exit orifices inserted through the proximal end of the previously placed peripheral catheter and advanced distally. There is a slider or plunger actuator coupled to the proximal exit orifice of the cannula to assist the insertion and retraction of the cannula to and from the previously placed peripheral catheter. Next, there is a step of coupling a fluid collection device to the lateral exit orifice of the housing, thus transferring fluid from the patient into a fluid collection device in an ergonomic and compact format. In some embodiments, the cannula proximal end is coupled to a distal end of a flexible tube. The flexible tube may include one or more coiled portions that are arranged in a spiral manner about the longitudinal axis of the cannula. A distal end of the flexible tube is in communication with a blood collection port in the handle or housing. The blood collection port may be positioned in any of a number of different positions advantageously depending upon the arrangement of the cannula and the slider. By way of example and not limitation, a blood collection port may be positioned in a sidewall or a proximal face of the handle, housing or protective structure.

In one embodiment, there is provided a blood extraction device having a housing with a proximal end and a distal end. There is a blood collection orifice in a blood collection port formed in a wall of the housing and a coupler disposed on the distal end of the housing, the coupler adapted and configured for engagement with a catheter or a peripheral vascular device. A slider accessible from outside of the housing and moveable in relation to the housing proximal end and the housing distal end. There is also a cannula having a proximal end and a distal end and a cannula lumen extending from a distal opening to a proximal opening, the cannula is within the housing and coupled to the slider wherein the cannula distal end is moveable beyond the coupler by axial movement of the slider, wherein the blood extraction device is in a first position when the cannula is inside of the housing and there is not a fluid pathway between the distal opening of the cannula and the blood collection orifice and the blood extraction device is in a second position when the cannula distal end is outside of the housing and there is a fluid pathway established between the cannula distal opening and the blood collection orifice. Additionally, the cannula lumen proximal opening is in a sidewall of the cannula, or the cannula lumen proximal opening is at the end of a curved proximal section of the cannula. In some embodiments, there may be a fluid path sealer within the cannula lumen wherein a distal end of the fluid path sealer is adjacent to the lumen proximal opening in the sidewall. In some embodiments, there is also a protective sheath around the cannula that is in an expanded condition when the blood extraction device is in the first position and is in a compressed position with the blood extraction device is in the second position. In other aspects, when the protective sheath is in the compressed position the sheath is distal to the blood collection orifice or proximal to the blood collection orifice. All of the methods and apparatuses described herein, in any combination, are herein contemplated and can be used to achieve the benefits as described herein.

In an additional aspect, there is a blood extraction device having a housing having a proximal end and a distal end and a blood collection orifice in a blood collection port formed in a wall of the housing. There is a coupler disposed on the distal end of the housing, the coupler adapted and configured for engagement with a catheter or a peripheral vascular device. A slider accessible from outside of the housing and moveable in relation to the housing proximal end and the housing distal end. There is a cannula having a proximal end and a distal end and a cannula lumen extending from a distal opening to a proximal opening, the cannula is within the housing and coupled to the slider wherein the cannula distal end is moveable beyond the coupler by axial movement of the slider. The blood extraction device is in a first position when the cannula is inside of the housing and the cannula lumen proximal opening is closer to the housing proximal end than the housing distal end and the blood extraction device is in a second position when the cannula distal end is outside of the housing and the cannula lumen proximal end spacing from the blood collection orifice is less than in the first position. In some alternatives, the cannula lumen proximal opening is in a sidewall of the cannula but in other variations the cannula lumen proximal opening coupled to a flexible tube section in communication with the blood collection orifice. In one alternative, the flexible tube section comprises a plurality of coils and the spacing between adjacent coils is greater in the first position than in the second position. There may also be a protective sheath around the cannula that is in an expanded condition when the blood extraction device is in the first position and is in a compressed position with the blood extraction device is in the second position or when the protective sheath is in the compressed position the sheath is proximal to the blood collection orifice. There is an aspect where the slider is configured as a plunger actuator extending from the proximal end of the housing wherein movement of the plunger actuator causes corresponding movement of the cannula lumen distal opening and the cannula lumen proximal opening while also changing a spacing between the cannula lumen proximal opening and the blood collection orifice. In one variation, distal movement of the plunger actuator reduces the spacing between adjacent coils on the plurality of coils in the flexible tube section.

In another alternative, there is a blood extraction device with a housing having a proximal end and a distal end and a blood collection orifice in a blood collection port formed in a wall of the housing proximal end. There is a coupler disposed on the distal end of the housing, the coupler adapted and configured for engagement with a catheter or a peripheral vascular device. There is also a fixed slider and a moveable slider accessible from outside of the housing, the moveable slider moveable in relation to the housing proximal distal end. There is a cannula having a proximal end and a distal end and a cannula lumen extending from a distal opening to a proximal opening, the cannula is within the housing and coupled to the moveable slider wherein the cannula distal end is moveable beyond the coupler by axial movement of the moveable slider, wherein the blood extraction device is in a first position when the cannula is inside of the housing and the blood extraction device is in a second position when the cannula distal end is outside of the housing and the cannula proximal opening and the blood collection orifice are closer to the fixed slider than when in the first position. In one alternative, there is a protective sheath around the cannula between the fixed slider and the moveable slider that is in an expanded condition when the blood extraction device is in the first position and is in a compressed position with the blood extraction device is in the second position. In some embodiments, the cannula lumen proximal opening coupled to a flexible tube section in communication with the blood collection orifice. The flexible tube section may include a plurality of coils and the spacing between adjacent coils is greater in the first position than in the second position or the flexible tube section comprises a plurality of coils and the spacing between adjacent coils is the same in the first position than in the second position. There may also be a spacing between the fixed slider and the moveable slider is less in the second position than in the first position.

In still another alternative, there is a blood extraction device having a housing having a coupler disposed at a distal end of the housing configured to engage a catheter assembly. There is a slider in communication with the housing, the slider configured to move between a distal end and the proximal end of the handle and a cannula comprising a distal end configured to extendable through the distal end of the handle when the slider is distally advanced in the handle. Further, a blood collection device in fluid communication with a proximal end of the cannula when the cannula distal end is extended into a blood vessel. There is an alternative where the cannula comprises a discontinuous lumen between the cannula distal end and the cannula proximal end that can be configured to transition from the discontinuous lumen to a continuous lumen when the cannula proximal end is aligned with the blood collection device coupled to an orifice on the handle. In another variation, the cannula comprises a discontinuous lumen having a linear segment distal to a flexible segment coiled within the handle interior, or the cannula comprises a discontinuous lumen having a linear segment extendable from the distal end of the handle and a flexible segment coiled within the handle interior, wherein the lumen is configured to direct blood flow in a first directed through the linear segment and in the opposite direction through the flexible segment. In these and other embodiments, there is also an orifice extending from the handle exterior to the handle interior and configured to couple with the blood collection device on the exterior of the orifice, wherein the cannula proximal end is in communication with the orifice. There may also be an orifice extending from the handle exterior to the handle interior and configured to couple with the blood collection device on the exterior of the orifice, wherein the cannula proximal end is in communication with the orifice. In another aspect, there may be a fluid transfer chamber configured to transfer blood between the cannula proximal end and the blood collection device, wherein the fluid transfer chamber is within the handle. In some embodiments, the slider is a plunger extending through the proximal end of the handle to the cannula. The blood collection device is a vacutainer assembly configured to couple with an orifice positioned on the handle and in fluid communication. In still other aspects, the cannula is configured to transition from the stowed position to an extended position with the cannula distal end extended beyond the handle distal end as the slider is distally advanced through the handle. In another aspect, the cannula comprises a lateral opening into a lumen extending between a proximal end and a distal end, the cannula opening configured to be aligned to fluid communication with the blood collection device when the slider advances the cannula sufficiently beyond the handle distal end. There may also be a fluid transfer chamber positioned at the distal side of the handle interior, wherein the cannula is advanceable through the fluid chamber through one or more seals configured to seal the fluid chamber as the cannula is extended or retracted through the handle to align the cannula lateral opening with the fluid transfer chamber interior. In still other aspects, the cannula lumen is configured to become continuous when the lateral opening is aligned with the fluid transfer chamber interior, or the cannula lumen is in fluid communication with the blood collection device along a length of deployment comprising length of the fluid transfer chamber within the handle interior.

In still other aspects, there is provided a blood collection device having a handle having a proximal end and a distal end with a hollow interior extending between the proximal end and the distal end and a coupler positioned at a distal end configured to engage a catheter assembly. There is a fluid transfer chamber at a distal end of the handle interior with a seal positioned on a proximal side and distal side of the fluid transfer chamber and a slider contactable from the exterior of the handle with a portion extending into the handle interior and configured to be distally advanced from the proximal side of the handle to the proximal side of the fluid transfer chamber. There is a cannula having a lumen extending between a proximal end and a distal end, the cannula axially aligned and extending from the slider through the fluid transfer seals and out of the distal end of the handle along with a lateral opening on the cannula between the cannula proximal end and the cannula distal end, wherein the lumen is configured to be advanced by the slider into a fluid communication with the fluid transfer chamber beyond the fluid transfer chamber proximal side to provide a continuous lumen from the cannula distal end through the lateral opening into the fluid transfer chamber and to a blood collection device in communication with the fluid transfer chamber.

In still another embodiment, there is a blood extraction device having a handle having a proximal end and a distal end with a hollow interior extending between the proximal end and the distal end with a coupler positioned at a distal end configured to engage a catheter assembly and an orifice extending through the handle with an engagement element on an exterior side of the orifice configured to engage a blood collection device. There is also a slider plunger contactable from the exterior of the handle and extending into the handle interior, the slider plunger configured to be distally advanced through the handle with a cannula having a lumen extending through a linear distal portion and through a flexible proximal portion in spiraled communication with the slider plunger segment within the handle interior, the cannula linear portion axially aligned with the handle and configured to be extended from the handle distal end to establish a flow of blood from a vessel when the slider is distally advanced within the handle. In some alternatives there are coils of the cannula flexible portion spiraled around the plunger are configured to compress when the slider is distally advanced to deploy the cannula distal end beyond the handle. In one alternative, the orifice is positioned at the distal end of the handle and the proximal end of the cannula is in fluid communication with the orifice. In another, the coils of the cannula flexible portion spiraled around the plunger are configured to elongate when the slider is distally advanced to deploy the cannula distal end beyond the handle. In yet another variation, the orifice is positioned at the proximal end of the handle and the proximal end of the cannula is in fluid communication with the orifice.

In still other alternatives, the various blood collection devices described above and herein may be advantageously used in a wide range of different methods of collecting blood from a patient blood vessel using via an in-dwelling catheter, based on the many unique attributes and design configures of the various embodiments. In one exemplary aspect there is a step of coupling a distal end of a blood extraction device of any of the blood extraction devices described above or herein to an indwelling catheter or other peripheral vascular access device in communication with a patient blood vessel. Next, there is a step of advancing a cannula distal end beyond the housing of a blood extraction device and into the patient blood vessel wherein the advancing the cannula distal end establishes fluid communication between the patient blood vessel and a blood collection orifice in the housing of the blood extraction device via a proximal cannula luminal opening. The blood is collected using the blood collection orifice in the housing.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 is an illustration of an intravascular blood extraction device according to an embodiment with an example of a cannula extended beyond the distal end of the protective housing and/or structures and/or structures, as described herein. The blood extraction device having a lateral exit orifice for connection to a fluid collection device.

FIG. 2 is an illustration of an intravascular blood extraction device according to an embodiment with an example of a cannula retracted within the protective housing and/or structures and/or structures, as described herein. The blood extraction device having a lateral exit orifice for connection to a fluid collection device.

FIG. 3 is an illustration of an intravascular blood extraction device according to an embodiment with an example of a laminar fluid path within the distal end of slider directly coupled to a cannula protected by a flexible and/or collapsible sheath disposed within the protective housing and/or structure, as described herein.

FIG. 4 is an illustration of an intravascular blood extraction device according to an embodiment with an example of the cannula extended distally beyond the distal end of the housing and/or structures, as described herein.

FIG. 5 is an illustration of an intravascular blood extraction device according to an embodiment with an example of a laminar fluid path within the distal end of slider directly coupled to a cannula protected by a flexible and/or collapsible sheath disposed within the protective housing and/or structure, as described herein.

FIG. 6 is an illustration of an intravascular blood extraction device according to an embodiment with an example of the cannula extended distally beyond the distal end of the housing and/or structures, as described herein.

FIG. 7 is an illustration of an intravascular blood extraction device according to an embodiment with an example of a laminar fluid path within the distal end of a plunger actuator directly coupled to a cannula protected by a flexible and/or collapsible fluid transfer tube disposed within the protective housing and/or structure, as described herein.

FIG. 8 is an illustration of an intravascular blood extraction device according to an embodiment with an example of the cannula extended distally beyond the distal end of the housing and/or structures, as described herein.

FIG. 9 is an illustration of an intravascular blood extraction device according to an embodiment with an example of a laminar fluid path within the distal end of slider directly coupled to a cannula protected by a flexible and/or collapsible fluid transfer tube disposed within the protective housing and/or structure, as described herein.

FIG. 10 is an illustration of an intravascular blood extraction device according to an embodiment with an example of the cannula extended distally beyond the distal end of the housing and/or structures, as described herein.

FIG. 11 is a cross section of an intravascular blood extraction device as illustrated in FIG. 8 with additional detail of a flow path through the lumen.

### DETAILED DESCRIPTION

An intravascular blood extraction device may include a cannula at least partially disposed within a housing and/or structures. The housing may also be referred to as a handle. The cannula can be configured to traverse the length of a previously placed catheter until access to a fluid vessel is achieved. Once inside of a vessel, it may be desirable for the cannula to advance beyond the distal end of the catheter into the vessel. A cannula structure of a single or a plurality of materials of a predetermined length may be selectively manipulated (e.g., advance or retracted) to a desirable location inside the vessel. The cannula structure may provide increased function, control, and effectiveness to the device.

An intravascular blood extraction device, as described herein, may comprise a cannula of single or plurality of materials oriented co-axially or circumferentially. For example, a stiffer cannula cross-sectional geometry with the use of a single or a plurality of materials. As described herein, a cannula may have a circumference comprising a soft and atraumatic outer polymeric layer, a lubricious inner layer compromising a segment of the laminar fluid path, and a stiffer layer within the two beforementioned layers to provide rigidity and column strength to the cannula. Wherein the stiffening layer, as described herein, may comprise of a single material or a plurality of materials to give the cannula the desirable rigidity.

In some examples, the co-axial stiffening segments within the outer and inner layers of the cannula may be positioned along the length of the cannula (e.g., stripped segments). Whereas the co-axial stiffening segments may encompass the total circumference of the cannula (0 to 360 degrees) or partially disposed within the circumference (e.g., less than 350 degrees, less than 340 degrees, less than 330 degrees, less than 320 degrees, less than 310 degrees, less than 300 degrees, less than 270 degrees, less than 180 degrees, or more). In some examples, more than one stiffening segment, as described herein, may be included along the length of the cannula so that the circumference of the cannula may be composed of the multiple stiffening segments.

In some examples, the stiffening layer of the cannula may be comprised of a softer distal end. For example, the softer distal end may provide an atraumatic tip insertion into the vasculature while maintaining the necessary rigidity and structure of the cannula proximal to the tip segment. Whereas the softer segment, as described herein, may have a predetermined length of at least 0.5 mm and extend proximally along the length of the cannula.

An intravascular blood extraction device, as described herein, may comprise of a cannula with a proximal end directly coupled to the entrance port of a plunger actuator. The plunger actuator, as described herein, located partially within the housing and/or structures and extending proximally outside of the housing and/or structures of the intravascular blood extraction device. The plunger has an entrance port in the distal end of the plunger actuator connected to the proximal end of the cannula. Whereas the entrance and exit port of the plunger are located in the distal end of the plunger actuator and form a laminar fluid path with the distal end of the cannula. The exit port of the plunger actuator is connected to the lateral top end of the intravascular blood extraction device with the use of a flexible transfer tube. Whereas the laminar fluid path of this embodiment is created by the fluid path from the distal end of the cannula extending within the fluid path formed between the entrance and exit port of the plunger actuator, and the flexible transfer tube directly coupled to the lateral top end of the intravascular blood extraction system. In some examples, the protective housing and/or structures is sealed to protect the cannula from contamination. The housing and/or structures is sealed distally around the cannula and proximally around the plunger actuator to prevent contamination of the cannula when in the first position. In some examples, the plunger actuator, as described herein, may move the cannulas from a first position within the protective housing and/or structures to a distal position outside the protective housing and/or structures. A distal end portion of the protective housing and/or structures may be configured with mechanisms for coupling the blood extraction system to a previously placed peripheral intravascular catheter or similar connector and/or connection.

An intravascular blood extraction device, as described herein, may comprise of a cannula protected by a collapsible and flexible sheath disposed within the housing and/or structures structure of the intravascular blood extraction device. The cannula directly coupled to the distal end of the slider and located within a flexible and/or collapsible protective sheath. A slider connected to the cannula and partially disposed outside the protective housing and/or structures and used to move the cannula from a first position inside the protective housing and/or structures to a second position outside of the intravascular blood extraction device. The flexible and/or collapsible protective sheath in the first position, may appear expanded, and contracted in the second position. The flexible and/or collapsible protective sheath maintains the sterile barrier around the cannula at all times.

A collapsible and flexible sheath having a distal and proximal end and coupled to the distal end of the protective housing and/or structures and the distal end of the slider. The slider moves the cannula from a first position within the protective sheath and/or structures within the housing and/or structures to a distal position outside the protective sheath and/or structures of the housing and/or structures. The flexible and/or collapsible protective sheath in the first position, may appear expanded, and contracted in the second position. The flexible and/or collapsible protective sheath maintains the sterile barrier around the cannula at all times.

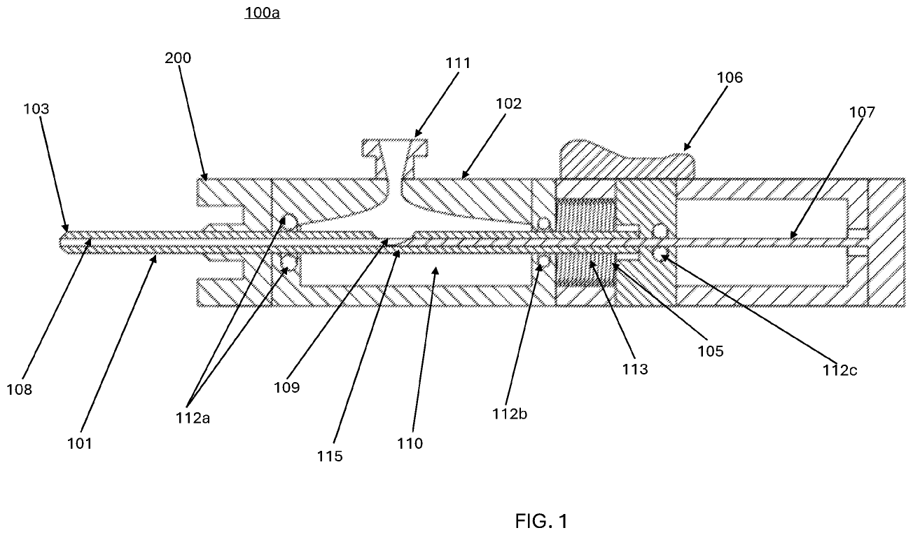

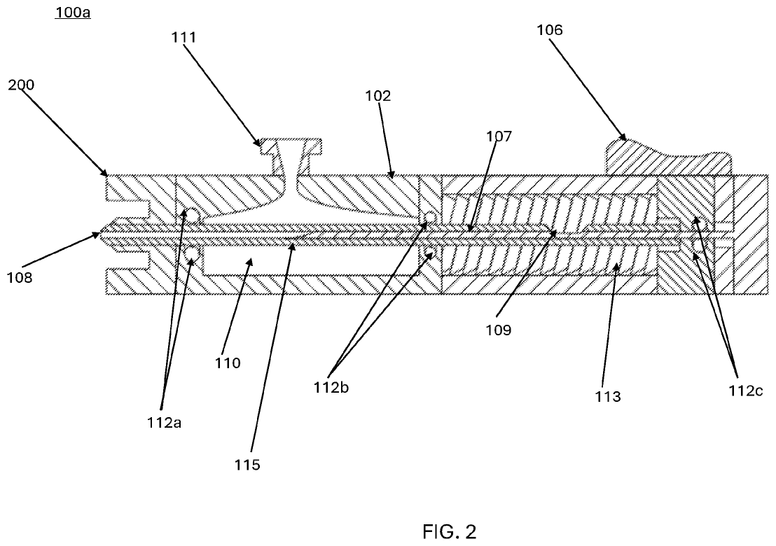

FIG. 1 illustrates an example of an intravascular blood extraction device 100a in its second and open laminar fluid path position, as described herein, having been extended forward thus the cannula 101 is partially disposed out of the protective housing and/or structures 102. An example of a blood extraction device with the cannula extended forward is illustrated where the cannula distal orifice 103 directly coupled to the distal end 105 of the slider 106 moved to the most distal position within the protective housing and/or structures. A fluid path sealer 107 is attached to the proximal end for the protective housing and/or structures to prevent backflow through cannula fluid path 108 until cannula proximal orifice 109 is within the fluid transfer chamber 110. The distal end 115 of the fluid path sealer 107 is aligned with the lateral blood collection orifice 111 as shown in FIGS. 1 and 2. With the cannula extended distally as shown in FIG. 1 the proximal opening of the cannula is aligned to the fluid sealer distal end 115. The fluid transfer chamber 110 is directly coupled to the lateral top orifice 111 thus forming the laminar fluid path intravascular blood extraction device. The fluid transfer chamber sealing around the cannula with seals, valves and/or gaskets 112a, 112b, and 112c to prevent leakage of fluid or vacuum pressure decay during use. Whereas the fluid path formed between the distal end of the cannula and the lateral orifice female luer has a continuing inner diameter of same dimension or a plurality of inner diameters of different dimensions to allow fluid to flow without hemolyzing or assist the fluid flow during use.

The lateral orifice 111 is a female luer where a fluid collection holder and/or syringe can be coupled. A flexible and/or collapsible protective sheath 113 is collapsed as the slider moves the cannula distal orifice from a first position inside of the protective housing and/or structures to a second position outside of the intravascular blood extraction device protective housing and/or structures. In this example, the fluid is extracted through the fluid path of the cannula through the proximal orifice of the cannula into the fluid transfer chamber. The transfer chamber is connected to a lateral top end device where fluid is collected (not shown).

The device 100a may include a protective housing 102 with a coupler 200 positioned on the distal end of the housing 102 and adapted to couple with a catheter or other vascular access component where the cannula 101 may be extended through and into a vessel. The slider 106 can be distally or proximally moved to adjust the cannula 101 deployment length. In some examples, as the cannula 101 is deployed, the slider 106 and slider distal end 105 compress the protective sheath 113 around the cannula 101 inside of the housing 102 and proximal to the fluid transfer chamber 110.

In some examples, the lumens of devices described herein may be discontinuous. As illustrated in FIG. 1 and FIG. 2, the cannula 101 has a lumen from the distal orifice 103 to the proximal orifice 109. Accordingly, the lumen from the distal orifice 103 is discontinuous and no fluid path is provided until the cannula 101 is deployed and the proximal orifice 109 is positioned within the fluid transfer chamber 109. In some examples, the fluid path sealer 107 can extend in the cannula lumen and prevent a flow of fluid proximally in the lumen beyond the distal end of the fluid path sealer 107. As shown in FIG. 1, the fluid path sealer is positioned within the canula 101 such that when the cannula 101 is deployed, the proximal orifice is not open to the fluid transfer chamber 110 until the cannula has been sufficiently deployed over the fluid path sealer 107.

The seals 112a, 112b, 112c are illustrated as cross section seals positioned within the protective housing 102 and around the cannula exterior to prevent flow of fluid on the outside of the cannula. There may be one or more seals positioned in and throughout the protective housing to prevent inadvertent fluid transfer through the device outside of the cannula. Some other examples of seals may include seals around the lateral top end 111 of the protective housing 102. The lateral top end (e.g., coupling feature) may be configured to engage with a vacutainer assembly for transfer of fluid from the cannula out to a container or receptacle such as a vacutainer.

FIG. 2 illustrated an example of an intravascular blood extraction device 100a in its first and closed configuration with the cannula 101 retracted within the protective housing and the proximal orifice 109 out of alignment with the fluid transfer chamber 110 with the position, as described herein, having a cannula initially disposed inside the protective housing and/or structures 102 and demonstrating an extended flexible and/or collapsible protective sheath 115 when the cannula is initially disposed inside the protective housing and/or structures and the slider is moved to the most proximal position of the intravascular blood extraction device. The protective sheath 113 is now in an expanded configuration with the slider 106 in the proximal position within the protective housing 102 with the cannula 101 and distal orifice 108 in a stowed configuration.

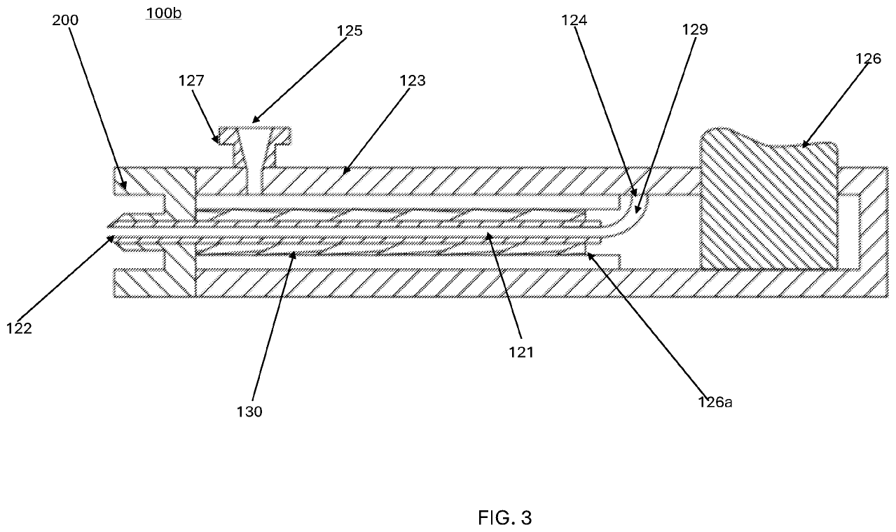

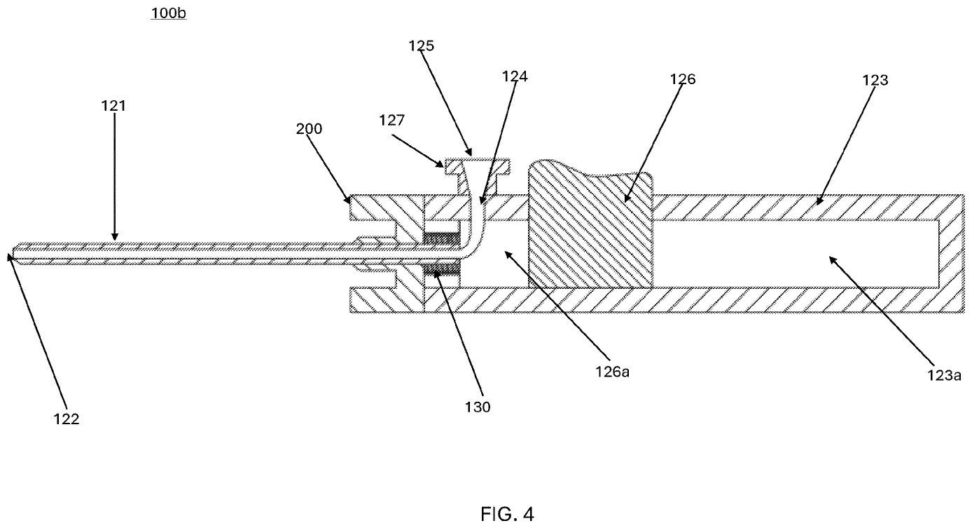

FIG. 3 and FIG. 4 illustrates examples of an intravascular blood extraction device 100b with cannula 121 with a distal end 122 in a first position inside of a protective housing and/or structures 123 and slider exit port 124. The slider exit port 124 can be coupled to a lateral orifice port 125 on the protective housing 123. The cannula 121 extends from the distal opening 122 to the slider exit port 129 with a curve 129 as it traverses the slider 126 through the slider distal end 126a. The slider exit port 124 can be fluidly coupled to the opening 125 thus allowing fluid to follow the laminar fluid path into the lateral orifice of the laminar fluid path when both ports are fully engaged. As described herein, the device 100b shows another example of a discontinuous lumen through the cannula 121 from the distal end 122 that me be deployed into a vessel and the proximal opening slider exit port 129 that is sealed or otherwise not in fluid communication with the lateral orifice 125 of the protective housing. This discontinuous lumen remains out of fluid alignment or communication with the lateral orifice 125 until the slider 126 is distally advanced and the cannula is moved distally to put the slider exit port 124 in fluid communication with the lateral orifice 125 through the coupler 127.

In some examples, the exit port of the slider 124 may communicate with the lateral orifice 125 which may comprise seals, valves, or gasket connections 127 to open when the slider 126 is coupled to the lateral orifice 125 of the intravascular blood extraction device in its most distal or forward position. The exit port of the slider 124, as described herein, having seals, valves, or gasket connections 129 to prevent leakage of fluid or vacuum pressure decay during use. The position or separation of these seals, valves, or gaskets may allow for the slider exit port to move into position and couple to the lateral orifice of the laminar fluid path.

In some examples, a cannula initially disposed inside the protective housing 123 through a protective sheath 130. In some examples, the protective sheath 130 can be collapsible as the slider 126 is distally advanced through the housing 123. As illustrated in FIG. 3, the device 100b is in a ready-to-use configuration or retracted configuration with the protective sheath expanded. For example, as illustrated, the device is in its first and closed laminar fluid path position and demonstrating an extended flexible and/or collapsible protective sheath 130 when the cannula 121 is initially disposed inside the protective housing 123 and/or structures and the slider 126 is moved to the most proximal position of the intravascular blood extraction device 100b.

FIG. 4 illustrates the device of FIG. 3 with the cannula 121 deployed beyond the distal end of the housing 123 through the coupling mechanism 200 with the distal opening 122 of the cannula 121 ready to direct a flow od blood through the cannula to a collection device (not shown) as the slider exit port 124 is now in fluid communication with the lateral orifice 125 and a flow can proceed through the cannula 121 out of the protective housing for collection. The slider 126 is in an advanced position within the handle 123 with the slider distal end 126a compressing the protective sheath 130. In some examples, the deployment length of the cannula 121 may be based on the length of advancement of the slider 126 through the handle interior 123a.

In some examples, a cannula in its second and open laminar fluid path position with the cannula extended forward and partially disposed out of the protective housing and/or structures demonstrating a compressed flexible and/or collapsible protective sheath 130 when the slider is moved to the most distal position of the intravascular blood extraction device.

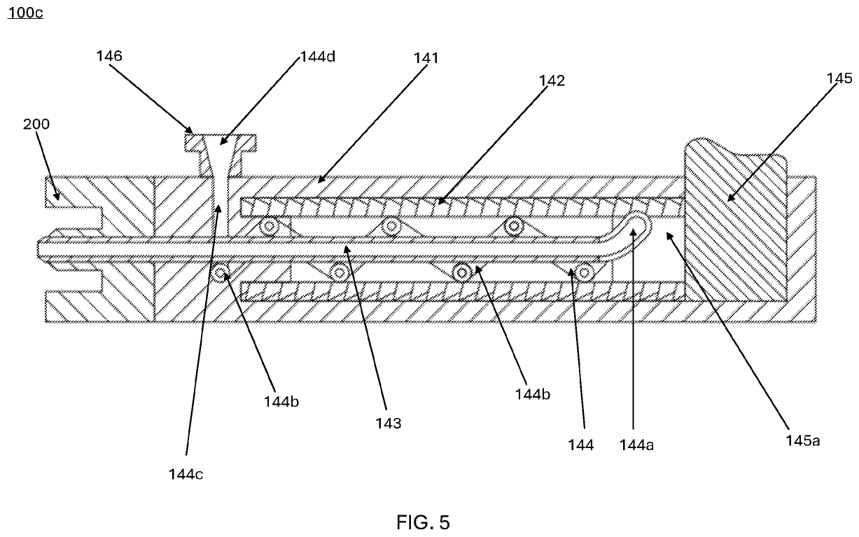

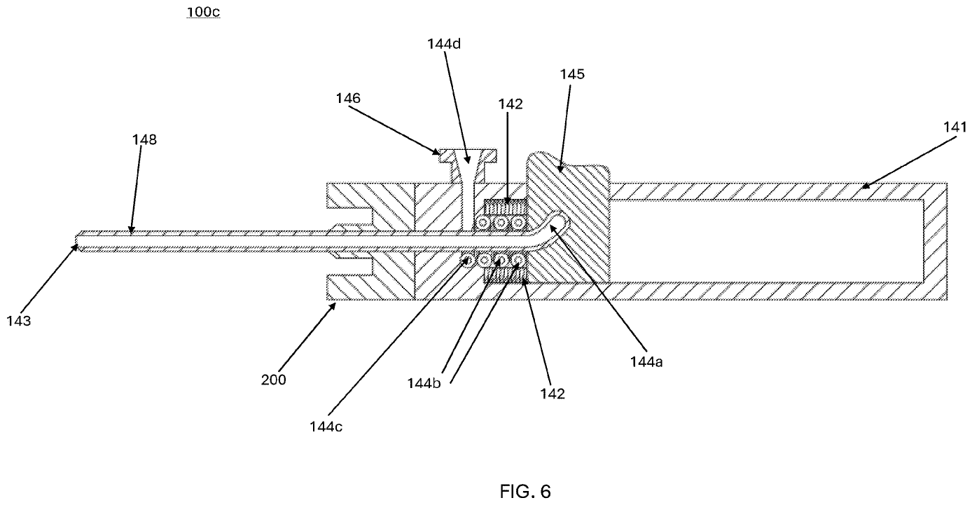

FIG. 5 and FIG. 6 illustrates an example of an intravascular blood extraction device 100c in its first position having a cannula 143 configured to provide a fluid path initially disposed inside of the protective housing and/or structures 141 and demonstrating an extended flexible and/or collapsible protective sheath 142 when the slider 145 is moved to the most proximal position of the intravascular blood extraction device 100c. The slider 145, as described herein, is disposed within the handle or protective housing 141 and/or structures to move the cannula from a first position within the protective sheath and/or structures 142 to a distal position outside the protective sheath and/or structures. A distal end portion of the housing and/or structures 200 may be configured with mechanisms of coupling the blood extraction system to a previously placed peripheral intravascular catheter or similar connector and/or connection. The slider 145 may be coupled to the housing and/or structures and/or coupled to the cannula and disposed outside and inside of the housing and/or structures of the device. In this cross section view, the slider 145, as other sliders described herein may extend through the protective housing with a portion of the slider on an exterior and engageable by a user with another portion of the slider within the protective housing 141 configured to manipulate the cannula 143 between a deployed and retracted configuration. In any of the examples described herein, the protective housing may be round or any other geometry allowing for the exemplary arrangement of features and elements described herein and also allowing for a sealable interior environment against inadvertent flow of fluids outside of the cannula.

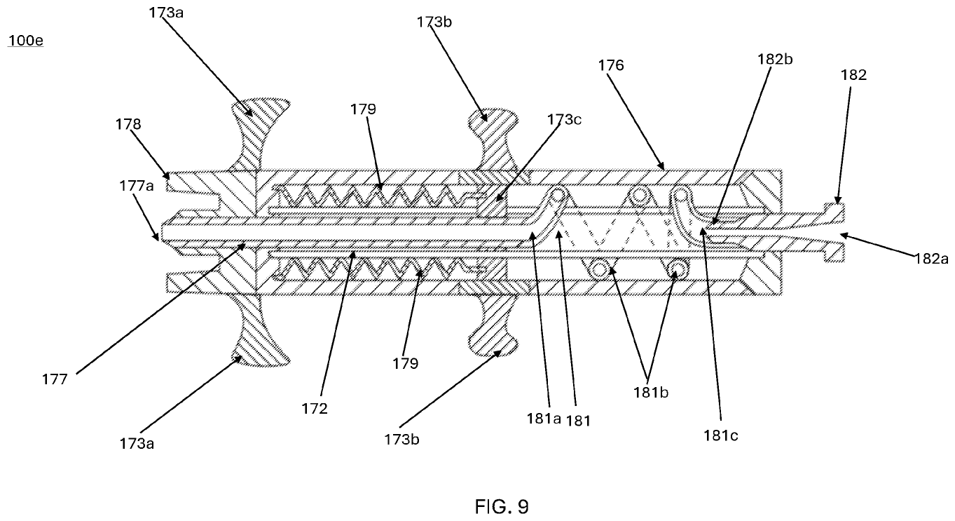

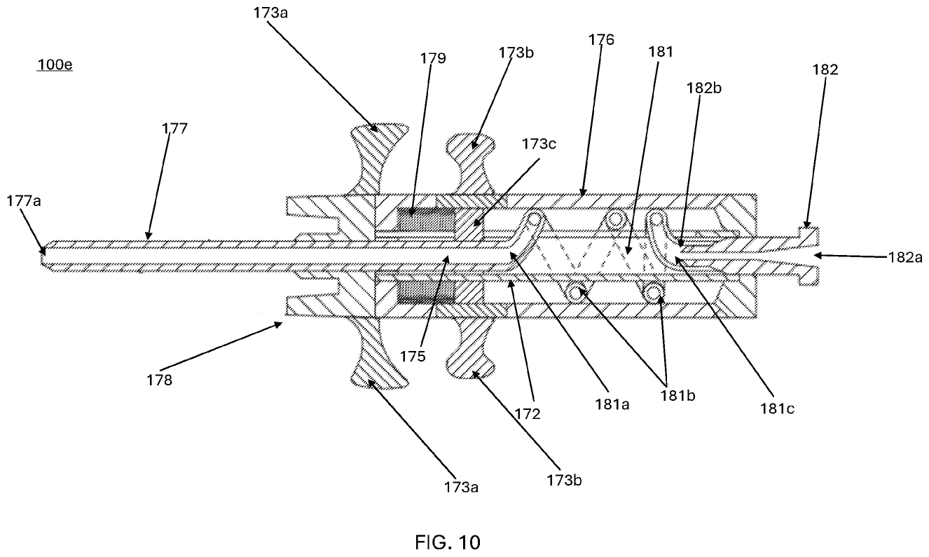

As described here, the devices comprise a discontinuous lumen. In some examples, the lumen may be discontinuous with an opening or other orifice (e.g., 109 in FIG. 1) that remains out of fluid communication with a port on the housing for a blood draw until the cannula is advanced to align the orifice with a fluid transfer chamber. In other examples, a lumen may be discontinuous as it relates to the configuration of the cannula through the device. For example, as illustrated in FIGS. 5 and 6, the discontinuous cannula 143 has a linear segment and a coiled or curved segment 143 that can be coiled or spiral around the linear portion of the cannula providing a reverse in the flow path direction from the cannula distal end to the lateral orifice or port 146 on the housing. The flexible tube 144 has a distal end 144a in communication with the proximal end of the cannula 143. The distal end 144a may be positioned within and moveable with the slider distal end 145a as shown in FIGS. 5 and 6. The distal end 144a has a curve, loop or bend that reverses the blood flow from the proximal portion of the housing distally toward the outlet 144d at coupler 146. Thereafter, the now distally directed flow moves along within the flexible tube 144 through one or more coiled segments 144b. The coiled segments 144b, in one embodiment, are arranged around the longitudinal axis of the cannula and/or housing to provide a pathway to a more distally located collection port 146. While coils are arranged for distal movement of flow in the embodiments of devices 100c and 100d, flexible coils may also be used to continue distal blood movement to a more proximal portion of the handle or housing as best seen in device 100e (see FIGS. 9 and 10).

In some examples, a fluid path forming a laminar fluid path and extending between the distal end of the cannula and the lateral orifice of the intravascular blood extraction device. The laminar fluid path, comprising of a cannula fluid path directly coupled to the distal end of the slider and a flexible transfer tube 144 connected to the distal end of the slider 145 and the lateral orifice of the intravascular blood extraction device. The flexible transfer tube 144b may be within the flexible and/or collapsible protective sheath 142. Whereas the fluid path formed between the distal end of the cannula and the lateral orifice female luer has a continuing inner diameter of same dimensions or a plurality of inner diameters of different dimensions to allow fluid to flow without hemolyzing or assist the fluid flow during use.

In some examples, a cannula initially disposed inside the protective housing and/or structures 141 in its first and closed laminar fluid path position and demonstrating an extended flexible and/or collapsible protective sheath 142 when the cannula is initially disposed inside the protective housing and/or structures and the slider is moved to the most proximal position of the intravascular blood extraction device.

In some examples, a cannula in its second and open laminar fluid path position with the cannula extended forward and partially disposed out of the protective housing and/or structures 148 demonstrating a compressed flexible and/or collapsible protective sheath 146 when the slider is moved to the most distal position 147 of the intravascular blood extraction device. Referring to FIG. 5, the device 100c is shown in a retracted or ready-to-use configuration with the cannula 143 retracted within the housing 141 and no flow path established through the linear segment of the cannula 143 or the coiled segment of the cannula. In FIG. 6, the slider 145 is now distally advanced with the cannula 148 deployed and the distal opening of the cannula 143 positionable within a vessel or through a catheter to preform a blood draw. The flow path may now be considered continuous as the fluid can flow through the linear segment of the cannula 143 then on and through the coiled segments 144a, 144b through to the coiled segment distal opening 144c and outlet 144d out of the protective housing through coupler 146 to a vacutainer or other collection device.

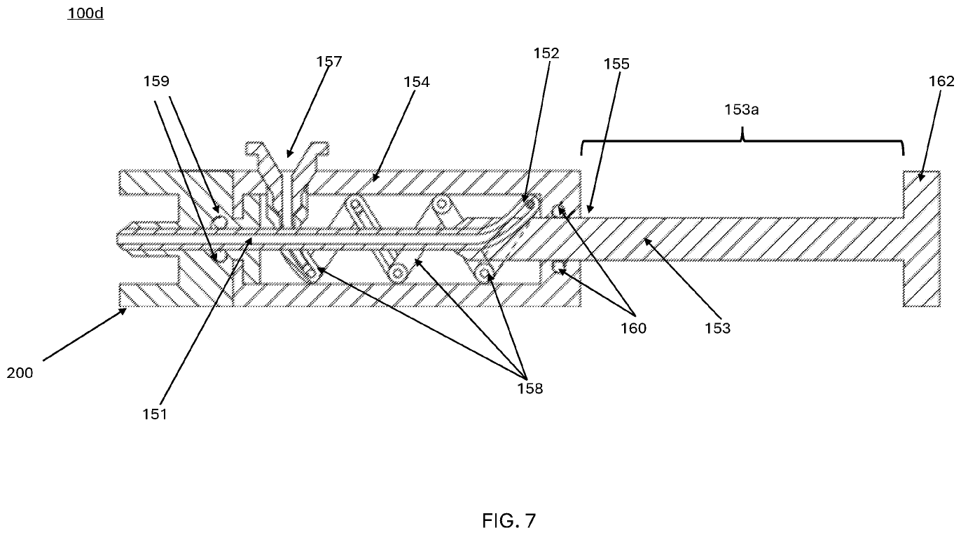

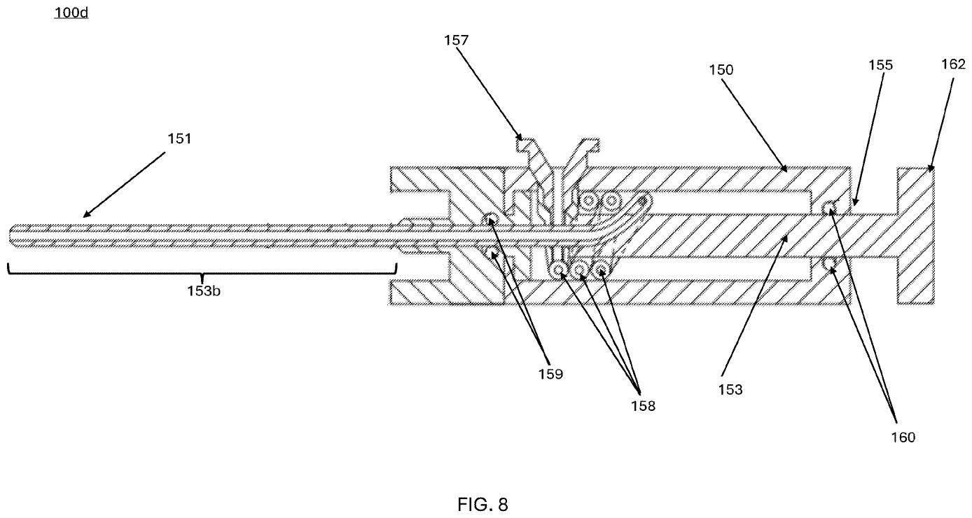

FIG. 7 and FIG. 8 illustrate an example of an intravascular blood extraction device 100d having a cannula 151 with a proximal portion 152 of the linear segment transitioning to the coiled segment 158 then out to the lateral orifice 157 on the handle or housing 154. In some examples, the cannula 151 may be directly coupled to the entrance port of a plunger actuator shaft 153. The plunger actuator 162 may extend from its proximal portion through the housing 154 with an actuator shaft 153. The housing shaft has an operable length 153a which is the amount of distal travel for the plunger actuator to move distally before making a hard stop at an outer wall of the housing or handle. The plunger distance 153a corresponds to the cannula travel distance 153b when the device 100d is in a blood collection configuration (see FIG. 8). In this view of the handle, housing and/or structures there are seen a number of sealed or protective enclosures for the cannula. The plunger has an entrance port 155 in the distal end of the plunger actuator connected to the proximal end of the cannula 152. In FIG. 7, the plunger actuator 162 is proximally retracted and the cannula 151 is in the stowed configuration within the housing 154. The linear segment of the cannula 151 is entirely within the housing 154 and the coiled or spiraled segment is expanded through the handle interior. The proximal end of the cannula is coupled to the lateral orifice or port 157 on the housing ready to be coupled with a fluid container during a draw.

In some examples, the plunger 153 may extend into the housing 154 through proximal seals 160 to seal the interior of the housing 154. The exit port 156 of the plunger (e.g., proximal end of the cannula) actuator is connected to the lateral top end 157 of the intravascular blood extraction device with the use of a flexible transfer tube 158 (e.g., coiled segment of the cannula 151). The transfer tube 158 is shown in its expanded configuration when the plunger is in its most proximal position. The laminar fluid path of this embodiment is created by the fluid path from the distal end of the cannula 151 extending within the fluid path formed between the entrance and exit port of the plunger actuator, and the flexible transfer tube directly coupled to the lateral top end of the intravascular blood extraction system. Whereas the fluid path formed between the distal end of the cannula and the lateral top end female luer has a continuing inner diameter of same dimension or a plurality of inner diameters of different dimensions to allow fluid to flow without hemolyzing or assist the fluid flow during use.

FIG. 8 shows the device from FIG. 7 now in a deployed configuration with the cannula 151 extending beyond the distal end of the housing 154 with a length 153b corresponding to the advancement of the plunger 162 through the housing 154. The cannula coiled segment or flexible segment 158 is shown as a more compressed coil still allowing for flow from the cannula distal opening to the port 157 on the housing in communication with the coiled segment of the lumen 158.

In some examples, the housing and/or structures is sealed distally 159 around the cannula and proximally 160 around the plunger actuator to prevent contamination of the cannula when in the first position.

In some examples, the plunger actuator, as described herein, may move the cannulas from a first position within the protective housing and/or structures to a distal position outside the protective housing and/or structures. A distal end portion of the protective housing and/or structures may be configured with mechanisms for coupling the blood extraction system to a previously placed peripheral intravascular catheter or similar connector and/or connection.

In some examples, a cannula in its second advanced position with the cannula extended forward and partially disposed out of the protective housing and/or structures 154 demonstrating a compressed flexible transfer tube 158 when the plunger 162 is moved to the most distal position of the intravascular blood extraction device.

FIG. 9 and FIG. 10 illustrate yet another example of an intravascular blood extraction device 100e with a cannula 177 again with a linear segment or region on a distal side and a coiled or spiral region 181 on the proximal side. The moving slider 173b can be used in combination with the fixed slider supports 173a for leverage when advancing the moving slider wall 173c distally to advance the cannula 177 from the handle or housing 176. The cannula 177 is coupled to the moveable slider wall 173c and deployed when the moving slider 173b is distally advanced towards the fixed slider support 173a. The flexible transfer tube or coiled segment 181 of the cannula can expand or contract according to the cannula deployment and retraction. A coiled segment distal end 181a is in communication with the proximal opening of the cannula lumen. Flow in the transfer tube 181 passes through the coiled segments 181b to the proximal end 181c. The coiled segment proximal end 181c is in communication with the outlet structures 182, 182a and 182b as best seen in the view of FIG. 10.

A distal end portion of the handle, housing and/or structures 176 includes a port or coupler 178 configured to couple the blood extraction system 100e to a previously placed peripheral intravascular catheter or similar connector and/or connection. The slider 173b may be coupled to and moveable relative to the housing and/or structures and the cannula and disposed outside and inside of the protective housing and/or structures of the device.

In some examples, a flexible and/or collapsible sheath 179 is directly coupled to the distal end of the protective housing and/or structures 176 (see connection adjacent to fixed slider 173a) and the moving slider 173b to prevent contamination of the cannula 177 prior to entering the previously placed peripheral catheter. The cannula, flexible or collapsible sheath and moving slider 173b may also be supported by a rail, wall or other suitable support structure, such as structure 172. The flexible and/or collapsible sheath 179 is extended state when the cannula 177 is in a first position within the protective housing and/or structures. (See FIG. 9). The flexible and/or collapsible sheath 179 is compressed in FIG. 10 when the cannula 177 is moved to a second position outside of the protective sheath and/or structures.

In some examples, a flexible transfer tube (e.g., coiled or spiral segment of the cannula) 181 can transition along with linear cannula segment and the moving slider 173b within the housing 176 as shown in the movement between FIG. 9 and FIG. 10. Optionally, the coiled segment 181 may also be configured to compress into a shorter axial spacing as in FIGS. 6 and 8. In some examples, the linear cannula segment 175 may extend from the distal opening 177a to the coiled segment 181 as it would transition through the slider 173b near region 175. The spiral or flexible segment of cannula 181 can then be coupled to the orifice or coupler 182 now positioned on a proximal end of the housing 176 with an opening 182a that can be in fluid communication with the cannula to allow for a flow of blood through the cannula 177 when deployed into a vessel or catheter assembly.

The example illustrated in FIG. 10 shows a laminar fluid path is formed between the distal end of the cannula 177a and the proximal end of the intravascular blood extraction device through the opening 182a. The flexible covering 179 has been compressed by the distal movement of slider proximal wall 173c shortening the overall length of the housing or handle and moving the collection port 182 closer to the distal end of the housing. In some examples, the proximal end of the intravascular blood extraction system may comprise of the coupler as a female Luer 182 or similar structure to couple a fluid collection device or syringe. There may be a distal opening 182b in some embodiments where the coupler 182 extends into the proximal end of the transfer tube 181. In some examples, the flexible transfer tube 181 is compressible and/or collapsible to allow the slider to move from a first position to a second position within the protective housing and/or structures 176. Whereas the fluid path formed between the distal end of the cannula and the proximal end female luer has a continuing inner diameter of same dimension or a plurality of inner diameters of different dimensions to allow fluid to flow without hemolyzing or assist the fluid flow during use.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

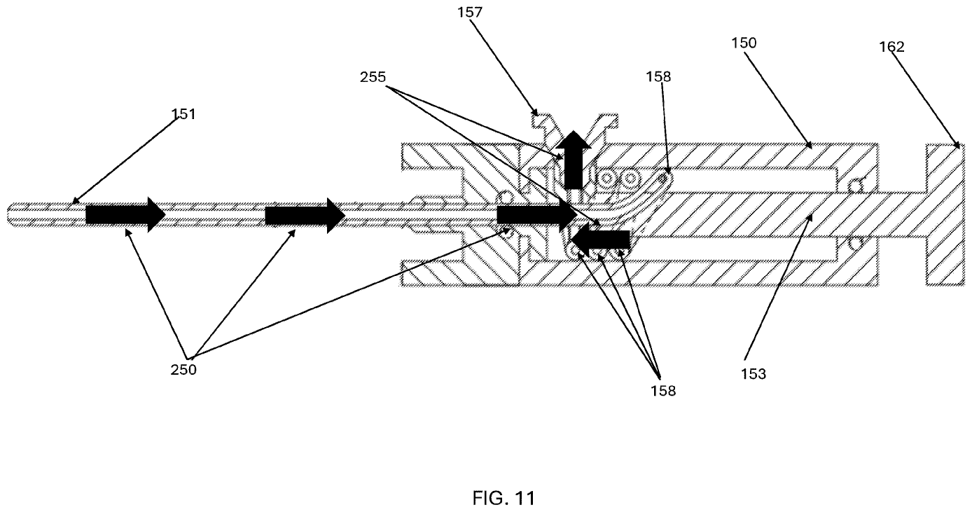

FIG. 11 shows a blood extraction device, as described herein, with arrows indicating an example of a flow path therethrough. This examples shows the device from FIG. 8 with the cannula 151 deployed as it may be within a vessel and the plunger 162 in the advanced position within the housing 150. A flow may proceed through the linear portion of the cannula 151 as indicated by arrows 250 and on through the coiled or spiraled section of flexible cannula tube 158 then on to the port or orifice 157 for transfer to a collection device such as a vacutainer. Arrows 255 indicated a reverse or change in flow direction from the linear portion as the blood may first travel in a proximal direction within the housing 150 then to the spiral segment of the cannula 151 in an opposite or distal direction to the coupler 157. The flow may be established when the lumen is continuous such that the flow is a singular flow that travels in a first direction, then to a second direction. In some examples, the flow through discontinuous lumens may travel in this opposite manner illustrated in FIG. 11. In some examples, the lumen may be discontinuous until the lumen is aligned with a fluid transfer chamber as illustrated in FIG. 1. In some examples, the discontinuous lumen may travel through a first linear segment of the cannula that is discontinuous as it transitions to a flexible spiral or coiled segment of cannula to the port or orifice on the housing.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A blood extraction device comprising:
a housing having a proximal end and a distal end;
a blood collection orifice in a blood collection port formed in a wall of the housing;
a coupler disposed on the distal end of the housing, the coupler adapted and configured for engagement with a catheter or a peripheral vascular device;
a slider accessible from outside of the housing and moveable in relation to the housing proximal end and the housing distal end; and
a cannula having a proximal end and a distal end and a cannula lumen extending from a distal opening to a proximal opening, the cannula is within the housing and coupled to the slider wherein the cannula distal end is moveable beyond the coupler by axial movement of the slider, wherein the blood extraction device is in a first position when the cannula is inside of the housing and there is not a fluid pathway between the distal opening of the cannula and the blood collection orifice and the blood extraction device is in a second position when the cannula distal end is outside of the housing and there is a fluid pathway established between the cannula distal opening and the blood collection orifice.

2. The device of claim 1, wherein the cannula lumen proximal opening is in a sidewall of the cannula.

3. The device of claim 1, wherein the cannula lumen proximal opening is at the end of a curved proximal section of the cannula.

4. The device of claim 2, further comprising a fluid path sealer within the cannula lumen wherein a distal end of the fluid path sealer is adjacent to the lumen proximal opening in the sidewall.

5. The device of claims 1-3, further comprising a protective sheath around the cannula that is in an expanded condition when the blood extraction device is in the first position and is in a compressed position with the blood extraction device is in the second position.

6. The device of claim 5, wherein when the protective sheath is in the compressed position the sheath is distal to the blood collection orifice or proximal to the blood collection orifice.

7. A blood extraction device comprising:
a housing having a proximal end and a distal end;
a blood collection orifice in a blood collection port formed in a wall of the housing;
a coupler disposed on the distal end of the housing, the coupler adapted and configured for engagement with a catheter or a peripheral vascular device;
a slider accessible from outside of the housing and moveable in relation to the housing proximal end and the housing distal end; and
a cannula having a proximal end and a distal end and a cannula lumen extending from a distal opening to a proximal opening, the cannula is within the housing and coupled to the slider wherein the cannula distal end is moveable beyond the coupler by axial movement of the slider, wherein the blood extraction device is in a first position when the cannula is inside of the housing and the cannula lumen proximal opening is closer to the housing proximal end than the housing distal end and the blood extraction device is in a second position when the cannula distal end is outside of the housing and the cannula lumen proximal end spacing from the blood collection orifice is less than in the first position.

8. The device of claim 7, wherein the cannula lumen proximal opening is in a sidewall of the cannula.

9. The device of claim 7, wherein the cannula lumen proximal opening coupled to a flexible tube section in communication with the blood collection orifice.

10. The device of claim 9, wherein the flexible tube section comprises a plurality of coils and the spacing between adjacent coils is greater in the first position than in the second position.

11. The device of claims 7-10, further comprising a protective sheath around the cannula that is in an expanded condition when the blood extraction device is in the first position and is in a compressed position with the blood extraction device is in the second position.

12. The device of claim 11, wherein when the protective sheath is in the compressed position the sheath is proximal to the blood collection orifice.

13. The device of claims 7-12, wherein the slider is configured as a plunger actuator extending from the proximal end of the housing wherein movement of the plunger actuator causes corresponding movement of the cannula lumen distal opening and the cannula lumen proximal opening while also changing a spacing between the cannula lumen proximal opening and the blood collection orifice.

14. The device of claim 13, wherein distal movement of the plunger actuator reduces the spacing between adjacent coils on the plurality of coils in the flexible tube section.

15. A blood extraction device comprising:
a housing having a proximal end and a distal end;
a blood collection orifice in a blood collection port formed in a wall of the housing proximal end;
a coupler disposed on the distal end of the housing, the coupler adapted and configured for engagement with a catheter or a peripheral vascular device;
a fixed slider and a moveable slider accessible from outside of the housing, the moveable slider moveable in relation to the housing proximal distal end; and
a cannula having a proximal end and a distal end and a cannula lumen extending from a distal opening to a proximal opening, the cannula is within the housing and coupled to the moveable slider wherein the cannula distal end is moveable beyond the coupler by axial movement of the moveable slider, wherein the blood extraction device is in a first position when the cannula is inside of the housing and the blood extraction device is in a second position when the cannula distal end is outside of the housing and the cannula proximal opening and the blood collection orifice are closer to the fixed slider than when in the first position.

16. The device of claim 15, further comprising a protective sheath around the cannula between the fixed slider and the moveable slider that is in an expanded condition when the blood extraction device is in the first position and is in a compressed position with the blood extraction device is in the second position.

17. The device of claim 15, wherein the cannula lumen proximal opening coupled to a flexible tube section in communication with the blood collection orifice.

18. The device of claim 17, wherein the flexible tube section comprises a plurality of coils and the spacing between adjacent coils is greater in the first position than in the second position

19. The device of claim 17, wherein the flexible tube section comprises a plurality of coils and the spacing between adjacent coils is the same in the first position than in the second position.

20. The device of any of claims 15-19, wherein the spacing between the fixed slider and the moveable slider is less in the second position than in the first position.

21. A blood extraction device comprising:
a housing having a coupler disposed at a distal end of the housing configured to engage a catheter assembly;
a slider in communication with the housing, the slider configured to move between a distal end and the proximal end of the handle;
a cannula comprising a distal end configured to extendable through the distal end of the handle when the slider is distally advanced in the handle;
a blood collection device in fluid communication with a proximal end of the cannula when the cannula distal end is extended into a blood vessel.

22. The blood extraction device of claim 21, wherein the cannula comprises a discontinuous lumen between the cannula distal end and the cannula proximal end that can be configured to transition from the discontinuous lumen to a continuous lumen when the cannula proximal end is aligned with the blood collection device coupled to an orifice on the handle.

23. The blood extraction device of claim 21, wherein the cannula comprises a discontinuous lumen having a linear segment distal to a flexible segment coiled within the handle interior.

24. The blood extraction device of claim 21, wherein the cannula comprises a discontinuous lumen having a linear segment extendable from the distal end of the handle and a flexible segment coiled within the handle interior, wherein the lumen is configured to direct blood flow in a first directed through the linear segment and in the opposite direction through the flexible segment.

25. The blood extraction device of claim 21, further comprising an orifice extending from the handle exterior to the handle interior and configured to couple with the blood collection device on the exterior of the orifice, wherein the cannula proximal end is in communication with the orifice.

26. The blood extraction device of claim 24, further comprising an orifice extending from the handle exterior to the handle interior and configured to couple with the blood collection device on the exterior of the orifice, wherein the cannula proximal end is in communication with the orifice.

27. The blood extraction device of claim 21, further comprising a fluid transfer chamber configured to transfer blood between the cannula proximal end and the blood collection device, wherein the fluid transfer chamber is within the handle.

28. The blood extraction device of claim 21, wherein the slider is a plunger extending through the proximal end of the handle to the cannula.

29. The blood extraction device of claim 21, wherein the blood collection device is a vacutainer assembly configured to couple with an orifice positioned on the handle and in fluid communication.

30. The blood extraction device of any of claims 21 to claim 29, wherein the cannula is configured to transition from the stowed position to an extended position with the cannula distal end extended beyond the handle distal end as the slider is distally advanced through the handle.

31. The blood collection device of claim 21, wherein the cannula comprises a lateral opening into a lumen extending between a proximal end and a distal end, the cannula opening configured to be aligned to fluid communication with the blood collection device when the slider advances the cannula sufficiently beyond the handle distal end.

32. The blood extraction device of claim 31, further comprising a fluid transfer chamber positioned at the distal side of the handle interior, wherein the cannula is advanceable through the fluid chamber through one or more seals configured to seal the fluid chamber as the cannula is extended or retracted through the handle to align the cannula lateral opening with the fluid transfer chamber interior.

33. The blood collection device of claim 32, wherein the cannula lumen is configured to become continuous when the lateral opening is aligned with the fluid transfer chamber interior.

34. The blood collection device of claim 32, wherein the cannula lumen is in fluid communication with the blood collection device along a length of deployment comprising length of the fluid transfer chamber within the handle interior.

35. A blood collection device comprising:
a handle having a proximal end and a distal end with a hollow interior extending between the proximal end and the distal end;
a coupler positioned at a distal end configured to engage a catheter assembly;
a fluid transfer chamber at a distal end of the handle interior with a seal positioned on a proximal side and distal side of the fluid transfer chamber;
a slider contactable from the exterior of the handle with a portion extending into the handle interior and configured to be distally advanced from the proximal side of the handle to the proximal side of the fluid transfer chamber;
a cannula having a lumen extending between a proximal end and a distal end, the cannula axially aligned and extending from the slider through the fluid transfer seals and out of the distal end of the handle; and
a lateral opening on the cannula between the cannula proximal end and the cannula distal end, wherein the lumen is configured to be advanced by the slider into a fluid communication with the fluid transfer chamber beyond the fluid transfer chamber proximal side to provide a continuous lumen from the cannula distal end through the lateral opening into the fluid transfer chamber and to a blood collection device in communication with the fluid transfer chamber.

36. A blood extraction device comprising:
a handle having a proximal end and a distal end with a hollow interior extending between the proximal end and the distal end;
a coupler positioned at a distal end configured to engage a catheter assembly;
an orifice extending through the handle with an engagement element on an exterior side of the orifice configured to engage a blood collection device;
a slider plunger contactable from the exterior of the handle and extending into the handle interior, the slider plunger configured to be distally advanced through the handle;
a cannula having a lumen extending through a linear distal portion and through a flexible proximal portion in spiraled communication with the slider plunger segment within the handle interior, the cannula linear portion axially aligned with the handle and configured to be extended from the handle distal end to establish a flow of blood from a vessel when the slider is distally advanced within the handle.

37. The blood extraction device of claim 36, wherein coils of the cannula flexible portion spiraled around the plunger are configured to compress when the slider is distally advanced to deploy the cannula distal end beyond the handle.

38. The blood collection device of claim 37, wherein the orifice is positioned at the distal end of the handle and the proximal end of the cannula is in fluid communication with the orifice.

39. The blood collection device of claim 36, wherein coils of the cannula flexible portion spiraled around the plunger are configured to elongate when the slider is distally advanced to deploy the cannula distal end beyond the handle.

40. The blood collection device of claim 39, wherein the orifice is positioned at the proximal end of the handle and the proximal end of the cannula is in fluid communication with the orifice.

41. A method of collecting blood from a patient blood vessel using via an in-dwelling catheter, comprising:
coupling a distal end of a blood extraction device of any of claims 1-40, to the indwelling catheter;
advancing a cannula distal end beyond the housing of a blood extraction device and into the patient blood vessel wherein the advancing the cannula distal end establishes fluid communication between the patient blood vessel and a blood collection orifice in the housing of the blood extraction device via a proximal cannula luminal opening.
